(19) United States
(12) Patent Application Publication
(10) Pub. No.: US 2025/0352628 A1
(43) Pub. Date: Nov. 20, 2025

(54) NON-TOXIC DESTRUCTION OF CANCER CELLS USING VIRAL VECTORED INJECTIONS

(71) Applicant: Richard Postrel, Miami Beach, FL (US)

(72) Inventor: Richard Postrel, Miami Beach, FL (US)

(21) Appl. No.: 19/280,080

(22) Filed: Jul. 24, 2025

Related U.S. Application Data
(62) Division of application No. 17/881,382, filed on Aug. 4, 2022.
Application No. 17/881,382 is a continuation-in-part of application No. 17/671,552, filed on Feb. 14, 2022.
Application No. 17/671,552 is a continuation-in-part of application No. 15/954,573, filed on Apr. 16, 2018, now abandoned.
Application No. 17/881,382 is a continuation-in-part of application No. 17/234,630, filed on Apr. 19, 2021.
(60) Provisional application No. 62/595,043, filed on Dec. 5, 2017.

(51) Int. Cl. A61K 39/00; C12N 15/86

(52) U.S. Cl. CPC A61K 39/001141; C12N 15/86; A61K 2039/5256; C12N 2760/16034; C12N 2760/16042; C12N 2760/16045

(57) ABSTRACT

The present invention provides a vector engineered virus designed to favor and infect cancers and surrounding cells. This technique mitigates cancer's ability to evade the body's immune system by calling attention to the cells that are cancerous beginning with their earliest stage of development by infecting those cells with a vector engineered virus specifically designed to target tumors in formation. This invention teaches a system and method for identifying, tagging, targeting, and destroying cancer cells while preserving healthy tissue. Developing cancers have two primary traits/biomarkers in common; one being a higher than normal heat signature from elevated metabolic activity; with the second being an acidic or lower than normal pH resulting from the hypoxic micro environment resulting from the depletion of available oxygen. Simply stated, all or most cancers have a heat signature greater than that of normal healthy cells and a pH lower than seen around normal healthy cells.

This application is a divisional following a Jul. 8, 2025 restriction in the parent application Ser. No. 17/881,382, filed 4 Aug. 2022 and through this application claims priority to U.S. application Ser. No. 17/671,552 filed 14 February-2022, 62/259,043 filed 5 Dec. 2017, Ser. No. 15/954,573 filed 16 Apr. 2018, Ser. No. 15/808,563 filed 9 Nov. 2017, Ser. No. 15/880,527 filed 25 Jan. 2018, Ser. No. 17/234,630 filed 19 Apr. 2011, 16/050,312 31 filed July 2018, Ser. No. 16/041,785 filed 21 Jul. 2018, PCT/US18/18650 filed 20 Feb. 2018, 62/460,997 filed 20 Feb. 2017, 62/482,192 filed 6 Apr. 2017, each of which is incorporated in its entirety by reference.

The present invention provides a vector engineered virus designed to favor and infect cancers and their surrounding cells by calling attention to the cells that are cancerous using a vector engineered virus specifically designed to target tumors in formation beginning with their earliest stage of development. This technique mitigates cancer's ability to evade the body's immune system by using the body's circulatory system to expose the virus to all the body's cells in search of their favored environment (hotter than normal cells with lower than normal pH factors). This invention teaches a system and method for identifying, tagging, targeting, and destroying cancer cells while preserving healthy tissue. Developing cancers have two primary traits/biomarkers in common; one being a higher than normal heat signature from elevated metabolic activity; with the second being an acidic or lower than normal pH factor resulting from the hypoxic micro environment resulting from the depletion of all available oxygen. Simply stated, all or most cancers cancers have a heat signature greater than that of normal healthy cells and a pH factor lower than normal healthy cells.

Unhealthy cells, including cancerous cells, signal their ill health to cells around them by intercellular or intracellular connections or more distantly through secretions into the circulation alerting and activating the body's immune system's defenses to eliminate the bad cells. But in cancers this system of signaling or of recognizing aberrant cells is greatly diminished or entirely disconnected allowing the aberrant cancer cells to survive and proliferate. This proliferation typically manifests itself to surrounding cells thereby spreading the cancer and accelerating its advance. This process enables the cancer's ability to grow by consuming and thus decreasing the nutrients available to proximal cells thereby enabling the cancerous tumor to evolve and enlarge.

This invention features a virus, such as an influenza derived virus, engineered and cultured to recognize cancer cells and to infect them and their surrounding neighbors. The virus may kill some infected cells by provoking that individual cell's inherent defenses. This viral infection will initiate systemic extracellular immune activities to fight infections in the cancer and in the locations immediate to the marked cancer cells.

This invention is enabled by vector engineering a flu virus. A flu virus, specifically and adaptively engineered to recognize the high temperature, low pH environments as cancerous targets for infection, selectively recognizes and infects the cells activating the body's immune systems to the site(s) of the cancer(s). This technique is universal to all cancers regardless of the type, location or stage of development. This unique use of a dual vectored virus creates a dominant infecting preference for locating at and attaching to cancer cells and those immediate to them. These engineered flu particles thus eliminate the need for chemotherapy, radiation therapy, and most major surgeries.

This invention is enabled by vector engineering a flu virus that is specifically and adaptively produced to recognize the high temperature, low pH environments of cancer as its target for infection. The engineered selectively recognizes and infects the cells to activate the body's immune systems at the site(s) of the cancer(s) and surrounding cells. This process encapsulates the cancer in formation eliminating it entirely.

To achieve this, a flu virus is selected and a first vector is grown in culture conditions to favor binding with cells at an elevated temperature. A second vector is grown in culture conditions to favor binding with cells at reduced pH. These engineering processes may be concurrent in the same culture (first and second vectors being the same) or may be performed in parallel. When the vectors are engineered in parallel, the two parallel cultures are then combined in culture to favor both elevated temperature and reduced pH. The co-cultured viruses reassort and adapt to produce a culture that favors both low pH and elevated temperature.

This technique is universal to all cancers regardless of the type, location or stage of development. This unique use of a dual vectored virus creates a dominant infecting preference for locating at and attaching to cancer cells and those immediate proximal to them. This engineered virus thus eliminates or greatly reduces the need for chemotherapy, radiation therapy, and most major surgeries.

The present invention activates destruction of cancer cells without harming the patient's immune system defenses. This technique requires the immune system be intact without radiation and/or chemotherapy suppressing the body's immune system to respond. The immune activating taggers of the present invention are delivered through the vascular system to overcome the cancer cells' avoidance of immune suppression. The flu vector targets cancer cells based on their inherent metabolic signature (rather than a mutated cell signature) so that the body's own natural immune recognizes the flu. Since the flu infection is targeted at a two component universal cancer cell metabolic signature the immune responses are directed to cancer cells wherever they are growing. Multiple levels of immune actions are involved including intracellular, extracellular, and chemical.

A biologic sensor that is engineered for preferential attraction to cells having the characteristic signature of cancer cells initiates cancer cell death by activating innate and acquired immune responses to the virus and virus infected cells at the site. The preferred vector is a virus, viral particle, or other engineered flu virus derived agent. The vector when bound at its target activates the organism's own innate immunity response systems and including responses to the flu virus adaptive immune systems at cancer sites. Where the flu virus infects one or more cells at the high temperature-low pH target sites and then self-replicates, the viral proliferation effect amplifies the local immune effect through the neighboring cells and encapsulates the tumor mass with a systemic immune response. In instance where the cancer cells have mutated to avoid both intracellular and systemic immune activation, non cancerous cells that are adjacent to the cancerous cells and thus in the zone of the cancer cell induced increased temperature and reduced pH, recruit an immune response when activated by the flu vector in the zone thereby encapsulating the cancer.

This comprehensive approach thus stimulates multiple inborn immune system signals which attract immune cells to the vector tagged cancer cell site. Once attracted to the cancer site, the immune cells and their chemicals carry out their normal defensive activities, which they are hard-wired to execute. The immune system is thus able to attack the flu virus vectored sites which include the infected and cancerous cells. Accordingly, this invention directs the immune system to eliminate the cancer and its potential to spread while sparing healthy tissues. This method is extremely effective in cases where internal portions of a tumor are not able to present or can only partially present an overt signal due to reduced or limited vascular development. This targeted immune attack completely surrounds the cancerous cells or tumor in formation, including their immediately adjacent cells, to completely encapsulate the cancerous cells or tumor in formation preparing them for destruction by the body's own immune system. In short this method allows the body to heal itself from cancers.

This method allows for weakly vascularized cells to succumb to immune attack as healthy cells along the periphery are used to call attention to the subject area to expose and eliminate the less accessible internal cancerous cells. This therapy reduces the need for radiation and/or chemotherapy and since it employs the body's own natural immune system. This technique is best employed prior to radiation and chemotherapies which comprise the body's own immune system.

### Cancer Development and Historical Therapies

Cancer succeeds because: i) cancer cells reproduce at a rate that far exceeds the growth rates of normal healthy cells, and ii) cancer cells elude the immune system that targets aberrant cells for elimination. As cells progress from a normal state towards a cancerous state, the cancer's uncontrolled growth requires more energy (than normal cells), consuming all available oxygen, and resulting in compensatory metabolic pathways. The accelerated metabolic rates and compensating metabolic pathways produce a heat signature higher than those of neighboring cells which release excess amounts of H⁺ (resulting in a locally reduced pH) and a unique universal cancer signature.

Cancer is not a single disease. Cancers arise in many different tissues, with a result that the plasma membranes of cancer cells do not express a cancer specific protein for universal recognition. One strategy has been to isolate an individual's cancer's cells in culture with the aim to form an antibody against that specific cancer to evoke an immune response targeting the cancer cells but sparing normal tissue. Numerous cultures each producing antibodies that react with normal tissue must be discarded while cancer specific antibody cultures are grown and tested for reactivity against other healthy tissues. This is an arduous process. The present invention provides an alternative to the cultures individualized for a specific cancer by engineering a virus that infects cells presenting these two universal features of cancer cells.

Although different cancers may appear in disparate tissues, and cancer cells may migrate from one tissue to another, at their root each cancer cell cohort involves a shift in normal metabolism from a lower to a higher metabolic rate, this shift being a universal characteristic of all hyperproliferating cancerous cells. As a cell transitions to become cancerous, it alters its metabolic pathways in various ways; down-regulating several, up-regulating others, possibly reinvigorating pathways used at an earlier time, for example during fetal development and turning off still others, such as those that limit cell growth, entirely. The present invention thus recognizes and can deal with cells that are not yet deemed cancerous but that have progressed substantially on a cancerous path.

Cancer cells are faster growing than non-cancer cells. The faster growth requires an accelerated metabolism with a greater number of chemical reactions that produce heat. The accelerated metabolism also shifts metabolism to a rapid pathway that produces the cell's energy source, ATP, with a byproduct of lactic acid. Thus, cancer cells exist in an environment where temperature is elevated and acidity is increased.

Regardless of the cell type originating the cancer or the stage of the cancer, cancer cells will present an increased uptake of nutrient building blocks into the cell to support growth—and increased use of the nutrients (reactants) in various chemical reactions to make increased products. The products will include products useful for sustaining the cell and by-products such as waste chemicals and heat. While there are some common chemical waste products of metabolism, one ubiquitous product (since in general metabolism is exothermic) is an increased heat output.

As an example of a changed metabolic requirement, each time a cell divides it requires its own set of nucleic acids to construct a second complete genome. To accomplish this, the nucleic acid production pathway must be up-regulated. But the up-regulation of one pathway requires diverting nutrient availability within the cell to deprive other pathways of their normal resource pools favoring transformation towards a more opportunistic cancerous supportive metabolic function. Outcomes of these metabolic shifts include an increased release of H⁺ with a resultant drop in pH and an increased release of small carbon containing molecules. This invention teaches vectored systems and methods for identifying, targeting and destroying cancer cells. As cells progress from a normal to a cancerous state their accelerated metabolic rates and adapted pathways generate amounts of H⁺ along with a higher heat signature that can serve as a targeting beacon for specialized cell killing vectors, e.g., a vector engineered influenza (flu) virus.

Influenza is a preferred virus at least because it frequently and continuously mutates to present variants no longer recognized by the previously infected immune system. These constantly appearing novel flu viruses require the updating and re-inoculation with flu vaccine which is done annually. When a flu virus is engineered to effect the present invention, a version that has not been recently active or is apparently novel to human infection should be selected to serve as a strain to be engineered. When a virus other than a flu virus is engineered, the seed strain should similarly be selected to avoid those that may have recently infected the target organism.

In the development stages, the cancer cell must intensify its metabolism to support the prolific growth and at the same time the transforming cell must debilitate the intracellular and systemic checks against uncontrolled cell growth that the body has developed to maintain homeostasis.

Cancer cells arise from diverse tissues and from many, many differentiated cell types, but at the root of all cancers is that cell's increased rate of making new cells, that is: hyperproliferation. Every time a cell proliferates it splits to create two cells—each of which requiring its own membrane, cytoskeleton, nucleus, mitochondria and other organelles. This duplication requires the cell to accelerate synthetic pathways and several additional pathways that support accelerated synthesis. The resulting two cells will require a doubling of DNA for duplicated nuclei, additional membrane lipids and proteins to cover the increased surface/volume ratio, extra endoplasmic reticulum, golgi, mitochondria, lysosomes, etc. to be split between two cells during mitosis. Mitosis itself is a resource hungry process requiring a slew of catabolic and anabolic events. In essence, a metabolic push is necessary to provide an additional set of all cellular components and the temporary resources and energy necessary to divide the cell into two. This accentuated metabolism results in increased biochemical reaction rates with increased exothermic heat production. This property can be employed to guide intercourse between the hyperproliferating cells and one or more vectors engineered to preferentially bind under these conditions.

Another important feature common to the metabolic shift of cancer cells is the decreased reliance on the ETC for making high energy phosphates, e.g., adenosine triphosphate (ATP). In order to make the ATP that is required in amplified amounts to support the increased metabolism that supports the hyperproliferation, cells switch on their back-up metabolic paths to emphasize a glycosylation process that ends with lactate (−) and hydrogen ion (H⁺) as byproducts. The additional H⁺ ions depress the pH (a measurement whose numeric expression decreases with increased H⁺ concentration). Another common byproduct of the glycosylation process is an increased abundance of various reactive oxygen species (ROS) such as H₂O₂ and. O₂.

The metabolic shift underlying increased metabolism deemphasizes the production of ATP through the electron transport chain (ETC). Pyruvate is not fed into mitochondrial metabolism, but rather is converted to lactate and transported chiefly by monocarboxylate transporter 4 (MCT4) wherethrough H⁺ and lactate (lactic acid) are delivered to the cell's exterior space. The H⁺ thus transported results in a local decreased pH that coexists with the increased temperature surrounding the cancer.

The aversion of cancer cells to the ETC and the conventional oxidative phosphorylation pathway is considered a requirement, not an anomaly of cancer cells. Remember that these cells were once considered “normal” cells but in their progression to the hyperproliferative state have had to alter normal cell functions. The hyperproliferation would be expected to change many metabolic pathways to support the new activities. These abnormal pathways would be expected to require abnormal raw materials or amounts of raw materials in the nutrients consumed or in the metabolic intermediates necessary to sustain the new way of life for the cell. It is thus wise to think of the altered metabolism, not as a symptom of cancer, but as links in the causative chain.

Cancerous cells divide more frequently than normal cells and are produced and retained in an amount in excess of the optimal needs of the organism. Cancerous cells are not eliminated in the regular growth regulating processes in the body. Cells on the way to becoming cancerous, pre-cancerous or hyperproliferative cells, may form and grow at a pace in excess of the organism's needs, but exhibit some normal processes of cell maturation and death. Cells with the elevated growth rates will exhibit many metabolic characteristics similar to cancer cells, such as increased heat production and shedding acid, and so will be targeted by vectors, such as an engineered virus that focuses on cells in zones of elevated temperature and reduced pH.

### Natural and Augmented Immunity

The innate immune system has both intracellular and extracellular components. The lethality of the 1918 pandemic influenza virus has been associated with insufficient innate intracellular response and extreme levels of virus replication resulting in severe lung inflammation and prolific infiltration into the lungs of neutrophils and alveolar macrophages.

This severe outcome seems to be the result of an especially inefficient intracellular innate immune response to this subtype of influenza with increased reliance on extracellular immunity and resultant inflammation. The limited efficacy of the innate immune response against the 1918 virus probable resulted from the adaptation of the virus NS1 gene to suppress the IFN-α/B system thereby permitting the virus to reproduce without immune restraints.

Host cells recognize the invasion/internalization of viruses and respond with strong antiviral activities. Viruses initially activate the innate immune system, which recognizes viral components through PRRs. On the other hand, acquired immunity plays a major role in the responses to re-infection with viruses. Host PRRs detect viral components, such as genomic DNA, single-stranded (ss) RNA, double-stranded (ds) RNA, RNA with 5′-triphosphate ends and viral proteins.

Previous pandemic viruses crossed species barriers after acquiring mutations that changed the binding preference of the HA from avian-like α-2,3 Sialic Acid (SA), to human-like α-2,6 SA. Some recently identified subtypes of avian influenza viruses have caused limited human infections, but none have acquired the capacity for efficient and sustained transmission among humans, a key property of a pandemic virus.

Detection of viral components by RLRs and TLRs in immune cells activates intracellular signaling cascades. This elicits secretion of type 1 IFNs, pro-inflammatory cytokines and chemokines, and increased expression of co-stimulatory molecules such as CD40, CD80 and CD86. type 1 IFNs activate intracellular signaling pathways via a type 1 IFN receptor, and regulate the expression of a set of genes. The IFN-inducible genes, such as protein kinase R and 2′5′-oligoadenylate synthase, are involved in eliminating viral components from infected cells and inducing apoptosis of infected cells. type 1 IFNs are produced not only by innate immune cells, including Dendritic cells (DCs) and macrophages, but also by non-immune cells, such as fibroblasts.

Proinflammatory cytokines and chemokines are also critical for eliminating virus infection by provoking inflammation and recruiting innate and acquired immune cells. Co-stimulatory molecules are essential for the activation of T-cells.

Innate immune cells are mammalian cells that do not recognize pathogenic material (e.g., cancer cells, bacteria, viruses, and yeast) by expressing an antibody or a TCR on its cell surface. Innate immune cells expresses receptors (e.g., receptors on its cell surface) or proteins that bind to the Fc region of other antibodies that are bound to a pathogen and/or receptors that bind to PAMPs that are associated with pathogens and/or DAMPs that are associated with damaged or transformed cells. Non-limiting examples of DAMPs include nuclear or cytosolic proteins (e.g., HMGB1 protein or $100 protein), DNA or RNA, purine metabolites (e.g., ATP, adenosine, or uric acid), and glycans or glycoconjugates (e.g., hyaluronan fragments). Non-limiting examples of PAMPs include bacterial lipopolysaccharide, flagellin, lipoteichoic acid, peptidoglycan, double-stranded RNA, and unmethylated CpG motifs. Additional examples of PAMPs and DAMPs are known in the art.

Non-limiting examples of innate immune cells include mast cells, macrophages, neutrophils, DCs, basophils, eosinophils, and natural killer cells. Additional examples of innate immune cells are known in the art.

The vector(s) of this invention is (are) engineered to identify and bind cells expressing the intensified metabolic signatures required for cancer's growth, and then by inserting into the cell, to trigger natural intracellular defenses that, in responding to the vector, also prevent continuing metabolism of the cancer cell. In the absence of a foreign pathogenic or chemical (e.g., an allergen) stimulation the cell's immune responses remain dormant. The cancer recognizing flu vector of the present invention Activates or initiates dormant metabolic pathways that will, when activated, support eradication of the targeted cell through evolved defenses such as apoptosis. Several of the expression products induced in response to the vector entry into the target cell also unleash a systemic effect by migrating to the cell membrane where: a) they serve as tags or markers of the infected cell; and b) by releasing cytokines, guide powerful killing cells from the immune system to the tagged cell. These natural extracellular processes provide additional backup measures to complete the destruction and removal of the targeted cancer cell.

The cancer's ability to evade the body's immune systems is defeated by the present invention's power to “light up” or “highlight” cancer cells without tagging or marking uninvolved healthy cells thereby alerting the body's immune systems to respond specifically to the targeted area of highlighted cancerous cells. The present invention directs the body's immune system towards cancer cells previously unnoticed by the immune system. Inserting foreign (e.g., viral) genetic material into the cancer cells highlights the cancer cells to alert the immune system to action in the virally infected zone.

Viruses, including flu viruses, are not static. They constantly morph and continue to improve capacities to propagate more viral entities (evolution). In this process they modulate their methods for controlling the resultant host cell's virus supporting metabolisms. The virus must commandeer a host cell's synthetic processes to make more virus, but must limit infection in the host organism to allow the contagion to spread.

But as humans and other organisms continually adapt to minimize and therefore better survive viral invasion, viruses also adapt to continue viral propagation. Among the 11 proteins encoded by influenza virus, the NS1 protein has been shown to block the production of IFN in infected cells. Such adaptations of an influenza virus allow it to partially or completely evade host cell innate immunity. These survival adaptations are easily avoided in an engineered virus that is easily recognized as a foreign attacker.

In addition to these natural viral changes, man has directed and controlled viral changes affecting, for example, host cell recognized by virus, and other means of replication and dispersal. For example, Sander Herfst et al, in their paper: “Airborne Transmission of influenza A/H5N1 virus Between Ferrets” published in Science 2012 describe some available methodologies used for directed viral adaptation. In essence two main concepts guide the new viral creations: a) selecting conditions for the virus to self-select according to survival of fittest principles and b) introducing genetic material or mutations into the viral genome. They used both targeted mutagenesis and serial passaging to select viral substrains advantageously growing in the passage target cell:

“Using a combination of targeted mutagenesis followed by serial virus passage in ferrets, we investigated whether A/H5N1 virus can acquire mutations that would increase the risk of mammalian transmission. We have previously shown that several amino acid substitutions in the RBS of the HA surface glycoprotein of A/Indonesia/5/2005 change the binding preference from the avian α-2,3-linked SA receptors to the human α-2,6-linked SA receptors . . . Passaging of influenza viruses in ferrets should result in the natural selection of heterogeneous mixtures of viruses in each animal with a variety of mutations: so-called viral quasi-species.”

In a similar influenza engineering exercise, Ron Fouchier et al reported producing an engineered H5N1 virus with massively increased ability to spread amongst humans.

The selected/engineered genes featured in the present invention may result in a varied induction within target cells, e.g., with different timing of expressed cellular response proteins, amount of protein expressed, species of protein expressed, etc. The innate immunity, including, but not limited to: interferons, cytokines, lymphokines, peptidylglycan recognition proteins, pattern recognition factors, interleukins, TLRs, etc., of the cell is thereby controllable by infection with one or more selected/engineered virus. In several instances the description in this application will use the slashed version “selected/engineered” as a reminder of the equivalency of result regardless of the term conveniently used. The reader will understand that selection may be considered one version of engineering or a part of the engineering process and thus the terms will often be considered equivalent when one or other appears without its slashed partner.

Engineering may include culturing the virus in a host environment that adapts the lipid envelope to increase melding with a targeted host at higher temperature or to decrease melding at non-elevated temperatures. But one characteristic of many cancer cells is that as the cancer developed, the innate pathways in a cell that signal the systemic immune system to attack abnormal cells is inactive. With the present invention, cells on the periphery of a tumor, possibly not fully progressed to “cancer” are recognized as targets, possibly because their metabolisms have started to transform, but definitely because of the higher heat and decreased pH immediate to the tumor. Another ring of supportive cells immediately surrounding the cancer is also targeted because of the “cancer signature” local temperature increase and decreased pH. These cells immediately adjacent to the cancer will, when infected by the virus, be fully active in drawing a full immune response to the cancer site even when the cancer cells have mutated to evade recognition by the immune system. The viral particles themselves, concentrating in the area in and surrounding the cancer are recognized as alien (foreign to the organism) bio-material and will elicit another immune response separate from the infected cells' releases of immune chemokines. The activities of the virus and immune system responding to the viral infection/presence surround and encapsulate the tumor to attract and direct immune activity to the cancerous cells, precancerous cells, and a zone of healthy cells, e.g., about 1, 2, 3, 4, 5, or 6 cell diameters, adjacent to and surrounding the tumor to encapsulate the entire tumor in formation. Thus the anti-cancer treatment will result in an infection event. The targeted organism may, depending on the strength of infection, exhibit symptoms commensurate with infections caused by viral strains similar to the source strain used for engineering.

Co-infection of a cell, in the lab or in an organism, with two influenza viruses from different origins (e.g. avian and human), can result in mixing of the RNA segments from the two viruses and formation of a new virus with an altered chimeric genetic make-up. Such swapping of gene segments between viruses, i.e., genetic reassortment, is one mechanism by which new influenza viruses with pandemic potential may arise in nature, but also a mechanism useful in a laboratory for engineering and selecting viral particles with desired traits.

For example, an H5N1 bird flu has been engineered (or modified) to infect humans. A surrogate mammalian species served as the culture medium for the bird flu which rapidly adapted to increase its proliferative abilities—by achieving airborne transmission capability. The relevant mutations were then sequenced providing a tool for engineering this trait into other virus species or subtypes. Such manipulations are common selection and engineering tools that might be used for optimization, in some instances merely routine optimization of infective virions, especially for example in phage viruses. Normal cell chaperones can be augmented in engineered culture cells to provide an efficient tool for assisted engineering of viral vectors with desired target cells and courier traits.

Since the virus must contact the target cell before infecting it, recognizable features are used by viruses to attach to and gain entry into their targeted cell. Any surface feature including, but not limited to: a membrane protein, a meldable lipid blend, a specialized raft, a glycoprotein, and/or any portion or fragment thereof, etc., might be recognized by a targeting virus. Viruses may be engineered using molecular biology and/or mutated or adapted using for example serial culture to obtain viruses that recognize one or more selective feature.

Serial selection and/or other types of engineered virus or bacteria may serve as a source of proteins or the information for making or engineering proteins that can be incorporated in a liposomal membrane. While it is possible to favor orienting transmembrane protein particles so that a chosen portion predominates on the outer surfaces, simplified production with pseudorandom orientation will generally suffice given sufficient amounts of protein available for protein incorporation. Sufficiency requires only a small number of proteins to be exposed on the outer surface to bind the target moiety.

Genetic engineering is a rapidly developing art increasingly including post transcription mechanisms of action. Examples include chemically modified siRNAs or short interfering nucleic adds (siNAs) as revealed in US Patents and Patent Applications such as: 20160244760, 20160053269 RNA Interference Mediated Inhibition Of Gene Expression Using Chemically Modified Short Interfering Nucleic Add (siNA), 20170022146 Novel Low Molecular Weight Cationic Lipids For Oligonucleotide Delivery (SIRNA Therapeutics (Merck), now owned by Anylam); 20160331828, 20160317647 Nucleic add Vaccines, 9464124, 20160271272 Engineered Nucleic Adds And Methods Of Use Thereof, 20160244501 Polynucleotides Encoding Low Density Lipoprotein Receptor; U.S. Pat. Nos. 9,295,689, 9,271,996 Formulation and delivery of PLGA microspheres, 9254311 Modified polynucleotides for the production of proteins, 9283287 Modified polynucleotides for the production of nuclear proteins (ModeRNA Therapeutics); 20170044239 Phage-Displayed Antibody Libraries And Uses Thereof (Academia Sinica); 20170037431 In vivo Gene Engineering with Adenoviral Vectors (University of Washington); 20170044541 miRNAs Enhancing Cell Productivity (1-lochschule Biberach); 20170044555 Recombinant RNA Particles And Methods Of Producing Proteins (Synthetic Genomics, Inc.). Viruses may be specifically engineered for identified cancers and may benefit from improved targeting at cells expressing higher temperature or excreting exaggerated amounts of hydrogen ion. Bacteria, with their own RNAses and binding proteins also impact the host cell genome and ability to continue growth.

Several studies have demonstrated the localization of viral structural proteins in membrane rafts and the effects of raft-disrupting agents (mainly removing reagents and synthesis inhibitors of cholesterol) in the replication processes of several viruses, including retroviruses (Retroviridae), RNA viruses (classified into Picornaviridae, Caliciviridae, Astroviridae, Reoviridae, Flaviviridae, Togaviridae, Bunyaviridae, Coronaviridae, Rhabdoviridae, Arenaviridae, Filoviridae, Orthomyxoviridae, and Paramyxoviridae), and DNA viruses (classified into Parvoviridae, Papovaviridae, Adenoviridae, Herpesviridae, Hepadnaviridae, and Poxviridae). Orthomyxoviridae or flu virus has characteristics preferable in the present invention. The flu genera Alphainfluenzavirus, Betainfluenzavirus, Gammainfluenzavirus, Deltainfluenzavirus, and Thogotovirus, are preferred genera already presenting with adaptations that can naturally infect mammalian cells and, with the exception of Deltainfluenzavirus, including human cells.

Influenza virus, a member of the family Orthomyxoviridae that is an enveloped virus containing a genome comprising eight segments of negative-sense single-stranded RNA (ssRNA) has strains that are especially sensitive to pH for their target cell binding and thus can be used to preferentially target cells in low pH environs produced by cancer cells that skew metabolism towards lactic acid as a metabolic product. The presence of multiple segments facilitates reassortment.

Influenza is a lytic virus which rapidly kills the host cell when the offspring virus are released. Since flu is a lytic virus the host cell genome is immediately incapacitated so that the cell can no longer divide to form offspring cancer cells. This contrasts with retro viruses like herpes and HIV which follow a lysogenic cycle, inserting viral reverse transcribed DNA into the host genome while the host remains viable. When the host cell divides, the lysogenic phase retrovirus remains incorporated into both new cells' genomes. But eventually the viral DNA is activated to produce large quantities of new virus particles whereupon that host cell ruptures (is destroyed) as the new particles are released.

The orthomyxoviruses are exemplary as our common, but sometimes deadly flu virus. influenzas A (Alphainfluenzavirus), B(Betainfluenzavirus) and C(Gammainfluenzavirus) infect many warm-blooded vertebrates including mammals and birds. Genera D (Deltainfluenzavirus) viruses have been observed in farm animals, but not yet in humans. Subtypes of each of genera A, B and C will infect the human organism. Notable subtypes of A include, but are not limited to: H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, etc. These nanoparticles may appear more spherical or more rodlike in shape and are somewhat larger (50-120 nm spheres) than exosomal particles or as thin as 20 nm to as long as several hundred nm when rodshaped.

Orthomyxoviruses or flu viruses naturally undergo slow change through small genetic changes passed down to daughter generations, or abruptly, through a process called “reassortment” where larger genetic segments swap between viral strains to create a new viral entity. Slow change is inherent in viral replication since each genome is independently polymerized and viruses have no capacity to correct misreads during duplication. Severe misreads simply cannot promulgate another generation either because their genes or gene products are nonfunctioning or they are outcompeted or easily identified and eliminated by the host immune defenses.

Viral re-engineering has been a niche but is now a growing art. For example, Asokan et al, Nature biotechnology, volume 28:1, Jan. 2, 2010, 79-82, teaches reengineering the receptor ligand of adeno-associated virus, with special emphasis on a basic [non-acidic] hexapeptide stretch at positions 585-590. (Charge and/or polarity of a peptide segment correlates positively with its availability for binding.) The engineered adeno-associated virus is defective in replication, requiring coinfection with another virus such as adenovirus, HSV, etc.

Madigan and Asokan, Current Opinion in Virology, Volume 18, June 2016, Pages 89-96 summarizes progress in engineering adeno-associated viral binding character. The glycan surface having been mapped, with multiple serotypes identified, isolated and characterized, bases for selecting optimal adeno-associated vectors is well-developed. “A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.” Multiple exemplary re-engineering successes are briefly mentioned in the paper along with a summary statement: “A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.” In this and other peer reviewed papers the adeno-associated virus is set forth as an advantageous candidate for vector re-engineering.

Hemagglutinin on the surface of the flu virus is instrumental for binding to and infecting target host cells. The 2009 swine flu is particularly illustrative of this phenomenon. Hemagglutinin mutated to become more acid stable as this H1N1 virus shifted from swine to humans. This lowered the pH at which the flu hemagglutinin was activated. The activation process triggers an irreversible change in the hemagglutinin's shape that then fuses the virus and target cell. The pH of activation is known to vary amongst various flu viruses. Avian and swine viruses are generally activated at about pH 5.5-6.0 compared to a>two-fold higher [H⁺] or pH about 5.0 to 5.5 predominant for human flu viruses. In the context of the 2009 pandemic, H1N1 swine viruses which were previously activated at pH 5.5-6.0 mutated to become activated at pH 5.5 at the pandemic inception and as the pandemic progressed, the activation pH of the H1N1 pandemic virus declined to 5.2-5.4. This mutation process can occur naturally as pH of the target changes or for purposes of the present invention culturing susceptible cells at decreasing pH levels, where targets may be selectively cultured to decrease their pH ranges for survival and growth or by switching the target cell line if preferred. Lowering the activation pH of the hemagglutinin may be one means of selectively targeting cells that favor a more acidic metabolism.

Hemagglutinin proteins from different strains and subtypes vary in activation pH values with a range from ˜4.6 to ˜6.0. Hemagglutinin proteins from HPAI viruses normally exhibit an activation pH value at the higher end of the range ˜6.0, while human seasonal viruses have lower pH activation values, ˜5.0 or less. H5N1 influenza virus isolates cluster in a range of ˜5.3 to ˜5.9. For individual viruses grown in sequential culture genetic drift is an effective tool for directed mutation towards a desired activation pH range to match that of a target host cell. For example, in H1, H3, and H7 influenza viruses, mutations that alter the hemagglutinin activation pH have been associated with changes in virulence in mice.

Although H1, H2 and H3, and N1 and N2 are the common human infecting hemagglutinins and neuraminidases respectively, others may mutate to be compatible with human cells as hosts and able to cause human disease and death. For example, the recent outbreak of bird flu was H7N9 killing several dozens of humans, but apparently was not able to replicate in a form transmissible from human to human. In another example, such virus with lytic potential but lacking transmission between untreated humans in contact with the recipient is prepared as a pH and heat targeting lytic vector. Influenzas B and C may be cultured and applied in the invention for similar considerations. An influenza A H5N1 virus (another drifted avian virus though weakly transmissible to humans) apparently requiring thousands of copies to infect a human can be extremely pathogenic as it may occasionally drift.

Infection by influenza virus' hemagglutinin surface glycoprotein binds sialic acid-containing receptors on the plasma membrane of a target host cell. In general, H5N1 influenza virus hemagglutinin proteins bind preferentially to a (2,3)-linked sialosides. Whereas human-adapted influenza viruses bind preferentially a (2,6)-linked sialosides. A switch from a (2,3) receptor binding specificity to a (2,6) receptor binding specificity may be preferred in adapting avian influenza viruses for mammalian hosts.

Initial viral infection arises via endocytosis or by injection of viral proteins and genes directly into the cytoplasm, by fusion of the viral envelope or by destruction of the viral capsids. Transcription and replication of DNA viruses except poxviruses generally happens inside the nucleus, whereas those of RNA viruses occur in the cytoplasm. However, influenza viruses are exceptional as RNA viruses with at least a major genome duplication occurring after transport to the target host cell nucleus. Before, after and during the transport and duplication processes, the innate immunity of the cell can act on the viral proteins and vRNA.

After receptor binding and internalization during influenza virus entry, the hemagglutinin protein is triggered by low pH to undergo irreversible conformational changes that mediate membrane fusion, and initiation of cell lethal infection either through apoptosis or other cell death or through lytic release of virus.

As an illustrative example, a class A influenza virus, e.g., H3N2, is cultured in a receptive host cell. (The H refers to the form of hemagglutinin; the N refers to the form of neuraminidase; human viruses have been H1, H2 and H3 and N1 and N2; H1N1 and H3N2 are most common infectious forms in humans. About 20 hemagglutinins are known, while neuraminidases have been seen in over 100 varieties.) The pH is gradually decreased with subsequent passaging. Attenuation is monitored to assure the virus remains infectious to human cells other than the cultured cell strain. In a preferred embodiment attenuation is observed at normal pH, but infectivity remains at elevated [H⁺].

In the low-pH environment of the endosome, the hemagglutinin is activated by a conformational change triggering its membrane fusion activity. The viral membrane fuses with the limiting membrane of the endosome to release the nucleocapsid into the cytosol. flu virus delivery of genetic material is rapid. The total infection period—from docking onto the cell's surface to the RNA entering the cell nucleus—is two hours. Influenza A because of its ability to mutate by both antigenic drift and shift is a preferred type of influenza virus for engineering select mutations in furthering this invention. In a low pH environment, the pH stabilized viral particle may facilitate development or may take advantage of tunneling nanotubes to pass infectious RNA to neighbor cells without necessity for forming an envelope. The temperature is also increased in culture to affect the content of the viral envelope to favor assimilation into membranes at increased temperatures. Alternatively, the low pH stable virus is allowed to mix with liposomes with higher melting temperature to transfer the liposomic constituents to the viral envelope lipid coating.

The resulting infectious virus is again screened or tested for selective infection at depressed pH and elevated temperature. Such virus may be delivered to a patient as a treatment for cancer, to target hyperproliferating cells and/or as a prophylactic event to seek out and eliminate cancerous cells that have not yet been outwardly observed, such as being palpated as a tumor mass. Some portions of tumors enter a quiescent state, for example when encapsulated by extremely active cells on the periphery which may prevent adequate nutrients, 02, etc., from reaching internal cells. These cells are subject to attack and removal by the immune system when the peripheral cells are infected. The infected peripheral cells provoke a general inflammation mediated through cells' intrinsic immunity activities and innate immunity processes associated with interferon mediated paths. The initial responses may promote cell suicide limiting viral replication, but also releasing cytokines that draw inflammatory cells to the site. The inflammation inducing cells secrete antibiotic substances, especially active oxygen compounds including, but not limited to: superoxide, hypohalites, and hydrogen peroxide.

These toxins will impact local cells underneath the peripheral cells. Additionally, viral induced stresses will promote intercellular bridging in the form of tunneling nanotubes (TNTs) that will transmit anti-viral activities to the neighboring cells, up to about five or six cell diameters distant.

Influenza A viruses are especially capable of inducing the expression of cytokine and pro-apoptotic genes in infected cells. Pathogenicity, cell lethality, replication efficiency, and transmissibility of influenza viruses depend on both viral genetic and host factors.

Hemagglutinin protein binds receptors and mediates viral-cellular membrane fusion during viral entry is the primary antigenic target during infection. Hemagglutinin protein is a trimeric class I membrane fusion protein that sports in its ectodomain a membrane-proximal, metastable stalk domain that is capped with a membrane-distal receptor-binding domain. Hemagglutinin protein is readied for membrane fusion by cleavage of the hemagglutinin precursor into a fusion capable hemagglutinin1-hemagglutinin2 complex. Some H5 and H7 hemagglutinin proteins can be cleaved by intracellular furin-like proteases to elicit systemic virus spread with enhanced virulence of such highly pathogenic avian influenza (HPAI) viruses.

The invention recognizes that not every cancer cell will be successfully infected. However, universal infection is not a requirement for attracting immune cells and/or inducing caspace dependent programmed cell death e.g., through necroptosis, pyroptosis, apoptosis, or caspace independent programmed cell death, e.g., autophagy, paraptosis, mitotic catastrophe, etc. Intercellular communications through chemical messaging and/or direct cytoplasmic connections between cells may share small macromolecules such as nutrients, RNA, signal peptides, etc., and larger components such as organelles as large as mitochondria, but they also share organism protective cell elimination or killing factors, spreading cell death to neighboring cells perhaps comprising a spherical shape as large as five or six cell diameters in radius.

An initial stage of immunity occurs within the cell under attack by a foreign (pathogenic) genome. Pattern recognition receptors (PRRs) recognize pathogen-associated molecular patterns (PAMPs) on invaders to initiate both the near instantaneous intracellular innate and the delayed and lasting adaptive immune responses. Toll-like receptors (TLRs) comprise an important set of PRRs where TLR activation initiates induction of interferons (IFNs) and cytokines active in both innate and adaptive immunity. Humans have at least 10 TLRs (appropriately numbered TLRs 1-10). The various TLR proteins bind different type targets, for example, TLRs 1 and 2 are involved in bacterial infections through their recognition of lipopeptides (1) and lipopeptides, lipoproteins and glycolipids (2); TLR3 recognizes double stranded RNAs and thus is preferentially effective against viruses. TLRs 7, 8 and 10 are activated in the presence of ssRNAs, especially of the types found in influenzas. TLR7 and TLR8 especially recognize GU of AU rich sequences of ssRNA viruses such as the Orthomyxoviridae family that includes influenza virus.

Compared with seasonal influenza virus H1N1, highly pathogenic avian influenza virus H5N1 is a more potent inducer of TLR 10 expression. Influenza virus infection increases associated TLRs expressions which contribute to innate immunity through their sensing the viral infection. This leads to cytokine induction, especially proinflammatory cytokines and interferons. Since TLR 10 induction is more pronounced following infection with highly pathogenic avian influenza H5N1 virus compared with a less pathogenic H1N1 virus H5 influenzas are a preferred initiator of cell death.

In one intracellularly initiated innate immune response, pattern recognition receptors (PRRs) inside a cell detect specific viral components such as viral RNA or DNA or viral intermediate products and induce production and secretion of type I interferons (IFNs) (e.g., IFN-α, IFN-β, IFN-ε, IFN-K and IFN-Ω) and other pro-inflammatory cytokines (including, but not limited to: IL-1β, IL-1Ra, IL-4, IL-5, IL-6, IL-7, IL-8, IL-10, IL-13, IL-17, G-CSF, GM-CSF, TNF-α, IP-10, MCP-1, MIP-1α, MIP-1β, RANTES, CCL-2/MCP-1, CCL-4/MIP-1β, CXCL-8/IL-8, CXCL-9/MIG, and CXCL-10/IP-10, keratinocyte-derived chemokine, etc.) in the infected cells and other immune cells to turn on intracellular controls and to signal the body of an attack. [Type II INF-γ, released by immune cells attracted to the infection site, has a secondary effect of potentiating type 1 IFN activity and acting as a cytokine for leukocytes.] The innate response is activated within hours of infection and may last for as long as 7 days during a primary influenza infection as the adaptive immune response is being activated.

This innate immune response stage is especially provoked when viral, bacterial or fungal pathogens infect our cells. As one example, influenza virus induces chemokine and cytokine production by infected epithelial cells and monocytes/macrophages. The chemokines attract immune cells, including macrophages, neutrophils and natural killer (NK) cells to the infected location. These cells then release more cytokines, chemokines and other antiviral proteins which provide an additional general killing mechanism as they initiate the adaptive (pathogen specific) immune response.

Type I interferons (IFN-α/B) are major cytokines produced by the innate immune response. They are produced inside an infected cell and by chemokine-recruited immune cells outside the infected cell. These bind internal receptors of the cell that produced IFNs and, when released, plasma membrane receptors on neighbor cells where they induce an enhanced antiviral response. One important early action of IFNs is production of intracellular antiviral proteins that also inhibit protein synthesis in general. This slows all growth including viral reproduction and may initiate an apoptotic event.

These interferons also recruit monocytes/macrophages, T cells and NK cells to the site and also act as signaling molecules to warn nearby cells of the viral presence. This signal induces neighboring cells to increase the numbers of MHC class I molecules upon their surfaces and heighten the immune response. And in the adaptive response they assist maturation of antigen-presenting cells (APCs) and increase expression of major histocompatibility complex (MHC) class I and II molecules on these APCs. These actions, of course, ramp-up antigen presentation for the adaptive immune stage.

The interferon invoked NK cells have a tremendous role in the innate immune response against viral infections. The NK cells are a class of large granular lymphocytes that recognize virus-infected cells in a non-specific manner. Since most infectious viruses down-regulate MHC class I molecules on the surface of infected cells as a survival mechanism to avoid destruction by cytotoxic T Lymphocytes (CTLs), the NK cells counter this by sensing the depletion of MHC class I molecules and then work to induce the infected cell's demise by apoptosis. Apoptosis is a highly orchestrated mechanism whereby a cell disassembles itself into small packages. The apoptotic process includes release of chemokines that attract phagocytic cells such as macrophages to ingest and carry away the disassembled parts.

Influenza PB1-F2 with a serine at position 66 is especially adept at inhibiting type I interferon production. This PB1-F2 binds to and inactivates mitochondrial antiviral signaling protein (MAVS). PB1-F2 protein is also associated with the induction of apoptosis and has a synergistic effect on the function of influenza virus polymerases PA and PB2. PB2 can also bind and inhibit the interferon promoter stimulator 1 (IPS-1) that normally promotes IFN-production.

However, although H1N1 viruses may be effective for infecting human cells, previous exposures to similar H/N epitopes may compromise access to target cells. Accordingly, it is advised to be cognizant of recent flu outbreaks that may have produced antibodies and other humoral reservoirs that might neutralize specific cell lines.

For example, influenza viral protein NS1 serves to bind viral RNA with its RNA binding domain to shield it from contacting ssRNA sensitive TLRs and retinoic acid inducible gene-l (RIG-I) a protein recognizing dsRNA including looped ssRNAs that complementarily bind. When stimulated by binding RNA, the TLRs, RIGI, and the like, induce type I interferon production. Some NSI proteins also bind the tripartite motif-containing protein 25 (TRIM25) that works though the activation of RIG-1 NS1s apparently also can complex with RNA-dependent protein kinase (PKR) and inhibit it. Otherwise, PKR is activated by binding double-stranded viral RNA and causes translation arrest in the cell nucleus including inhibition of viral protein synthesis. As another defense, the influenza virus M2 protein can inhibit P581PK to inhibit protein synthesis, and arrest host cell apoptosis.

The viruses engineered for use in the present invention must possess capacity to infect cells and provoke an immune response. These viruses will preferably be attenuated to provoke adequate but not overwhelming immune attack. However, virus particles are capable of spontaneous mutation. Viruses also are known to swap genes and thereby change target cell, pace of infection, number of particles produced per infected cell, etc. Accordingly, preferred embodiments engineer viruses to incorporate at least one, but potentially a plurality of recognition sites for antiviral compounds, either existing and repurposed for this function or selected or designed specific to the binding site associated with the engineered virus. These recognition sites may also provide a two-tiered approach wherein the viral coat incorporates an engineered component that appears on the cell's membrane following fusion. This component can serve as a recognition site for elimination of the attacked cell, in some circumstances when the viral infection itself has not induced the robust immunogenic response to kill the cell and/or neighbor cells.

Any available targeting or delivery means known in the art can be used. For example, a viral particle can be engineered to deliver a therapy to the targeted cell's interior. In the example of a reovirus which infects cells that express an activated ras oncogene, the cell is rendered more prone to infection by the virus since the activated Ras system deactivates antiviral defenses the cell would normally use to prevent reovirus infection. An engineered retrovirus, like a reovirus, or other vector known in the art is therefore a viable courier for a variety of therapeutic strategies to modulate intracellular metabolism especially when anti-viral defenses are compromised as often occurs when a cell ramps up its proliferative capacity.

The present invention overcomes the cancer's ability to avoid the body's practice of eliminating improperly acting cells by attacking such cells with a flu virus engineered to preferably bind cells of any derivation that express the hyperproliferation induced excess heat and acidity inherent in cancer cells. A flu viral vector is adapted though molecular engineering or adaptive culture to preferentially bind a cell at elevated temperature compared to the organism's normal cell temperature. This vector or a second vector is adapted though molecular engineering or adaptive culture to preferentially bind a cell where the [H⁺] is higher compared to the organism's normal cell pH. A first and second vector can be co-cultured to reassort and acquire preferential binding for both elevated temperature and acidity. A vector so engineered to recognize these two universal traits of cancers upon infusion preferentially infects cancer cells and cells in the elevated temperature and increased acidity zones supporting the cancer cells. The flu infection then provokes natural immune responses against the flu but also the cancer that heretofore had been avoiding elimination by the immune system.

Engineering is an arbitrary term that may include targeted mutagenesis, selection mutagenesis, motif swapping, gene swapping, capsule or envelope substitution, etc. A virus, for example, an RNA virus, may be mutated to incorporate a sequence from another virus and possibly packaged in a coating co-produced during viral replication with a DNA virus. The viral type name may thus be arbitrarily based on the viral component relevant to a desired, selected, engineered, mutated, etc., activity.

I claim:

1. A method for eliciting a natural immune response to selectively attack cancerous cells while sparing normal, non-hyperproliferating, cells, said method comprising: a) selecting a first virus of the Orthomyxoviridae family as a first vector; b) selecting a second virus of the Orthomyxoviridae family as a second vector; c} engineering said first virus to increase selective binding to a cell presenting with a temperature elevated in comparison to the cells producing said first virus to produce said first vector; d) engineering said second virus to increase selective binding to a cell presented in a pH depressed in comparison to the cells producing said second virus to produce said second vector; e) co-culturing said first vector and said second vector to produce a third vector with increased selective binding to a cell presenting with a temperature elevated in comparison to the cells producing said first virus to produce said first vector and selective binding to a cell presented in a pH depressed in comparison to the cells producing said said second vector; f) presenting said third vector for infusion to selectively attack cancerous cells.

2. The method of claim 1, wherein infusing said third vector producing said innate immune response induces cytokine production at the site of the hyperproliferating cells.

3. The method of claim 2, wherein said cytokines comprise Type 1 interferon.

4. The method of claim 1, wherein said first virus is a flu virus.

5. The method of claim 1, wherein said second virus is a flu virus.

6. The method of claim 1, wherein said first virus is a flu virus novel to humans, one not active in recent vaccinations and said second is a flu virus novel to humans, one not active in recent vaccinations.

7. The method of claim 1, wherein said pH is depressed in accord with a greater than 2 fold higher [H⁺].

8. The method of claim 1, wherein at least one of said first virus and said second virus is a H3N2 influenza virus.

9. The method of claim 1, wherein at least one of said first virus and said second virus is a PB1-F2 with a serine at position 66.

10. The method of claim 1, wherein said engineering comprises a process selected from the group consisting of: targeted mutagenesis, selection mutagenesis, motif swapping, gene swapping, capsule, and envelope substitution.

11. The method of claim 1, wherein at least one of said first virus and said second virus is an Influenza A.

12. The method of claim 1, wherein infusing said third vector producing said innate immune response recruits a lymphoid cell selected from the group consisting of: dendritic cell, monocyte, macrophage, NK cell, and T-cell to the attack site.

13. The method of claim 1, wherein infusing said third vector producing said innate immune response recruits dendritic cells, monocytes, macrophages, NK cells, and T-cells to the attack site.

14. The method of claim 1, wherein the selective attack is independent of originating cell type of the cancer.

15. The method of claim 1, wherein the selective attack is independent of stage of the cancer.

16. The method of claim 1, wherein the selective attack is independent of location within the body of the cancer.

17. The method of claim 1, wherein said human comprises cancerous cells, said cancerous cells being said hyperproliferating cells.

18. The method of claim 1 further comprising:
g) delivering said third vector into a human to elicit a natural immune response that selectively attacks hyperproliferating cells while sparing normal, non-hyperproliferating cells.
